(19) United States
(12) Patent Application Publication
(10) Pub. No.: US 2002/0120243 A1
(43) Pub. Date: Aug. 29, 2002

(54) Intravaginal device for feminine hygiene

(76) Inventors: Robert Kraemer, Koeln (DE); Herve Buzot, North Brunswick, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/027,715

(22) Filed: Dec. 20, 2001

Related U.S. Application Data
(60) Provisional application No. 60/305,656, filed on Jul. 16, 2001.
(60) Provisional application No. 60/257,871, filed on Dec. 22, 2000.

(51) Int. Cl. A61F 13/15

(52) U.S. Cl. 604/367

(57) ABSTRACT

The invention relates to an intravaginal device for feminine hygiene that comprises a core which is completely surrounded by a sheathing that is impermeable to liquid water.

### FIELD OF THE INVENTION

The present invention relates to an intravaginal device for feminine hygiene.

### BACKGROUND OF THE INVENTION

Intravaginal devices may include absorbent tampons for menstrual use, medicated tampons for delivery of medicaments, contraceptive devices, collection cups for menstrual use, urinary incontinence devices for bladder support, and the like.

Urinary incontinence, for simplicity referred to hereafter as incontinence, is a widespread problem among women of all ages, from which they suffer either periodically or permanently. In addition to physical damage or deformities, which can only be eliminated surgically, incontinence is often triggered by stress or physical exertion. This often occurs when tissue and muscles supporting and controlling the urinary bladder are weakened and/or displaced in the body cavity.

To counteract incontinence of this kind, various aids supporting or bearing the neck of the urinary bladder are known in the form of intravaginal incontinence devices that support the neck of the urinary bladder and, in particular, keep it closed during physical movement and/or sporting activity.

WO 95/05790 discloses a vaginally insertable incontinence device which is produced from a shaped body of compressible, elastic material. The shaped body is humidified before it is deformed and inserted into the body cavity. Furthermore, the shaped body is of a special form, which comprises a flexible central part and two legs, which extend away from the central part. By means of a rod-shaped applicator, which is placed against the flexible central element, the incontinence device is positioned in the vagina in such a way that the neck of the urinary bladder is supported on account of a spreading of the legs of this device induced by the elasticity of the material. The complex form of this device makes exact positioning of the same in the body cavity more difficult. The incontinence device inserted into the body cavity may, however, change its position as a result of body movements of the user, so that an effective, permanent raising of the neck of the urinary bladder may only be achievable to a restricted extent or not at all.

In WO 88/10106 there is described an essentially cylindrical, female urinary incontinence device of foam material. This known incontinence device must likewise first be brought into an elastic state by impregnating with liquid to allow it to be inserted into the body cavity. A polyvinyl foam is specified as the only unrestrictedly suitable material. This incontinence device has a large diameter and is easily compressible after impregnating with water, so that reliable, troublefree insertion and positioning of this intravaginal device is not ensured.

### SUMMARY OF THE INVENTION

The object of the invention is to provide an intravaginal device for feminine hygiene which needs no preparation before insertion into the body cavity and has a high degree of rigidity and dimensional stability, so that when needed it can be inserted by the user immediately and essentially without any trouble and can be accurately positioned permanently. At the same time, the intravaginal device is to have a high positional stability while being worn, even during physical exertion of the user. Furthermore, on the basis of its form and size, the intravaginal device is to convey a positive feeling and permit cost-effective production.

According to the invention, both the geometrical form and the geometrical dimensions of the intravaginal device as well as the position of the same in its state of having been inserted into the body cavity are largely retained even when the user is engaging in vigorous physical movement or under great stress and under the compressive forces thereby exerted on the intravaginal device. The intravaginal device according to the invention can be used without previously having to be brought into a usable, i.e., insertable, state by impregnating with liquid. Furthermore, the intravaginal device has relatively small dimensions, so that the intravaginal device can be inserted well into the body cavity and can be positioned accurately and permanently, and a consistent effect is achieved even when it is used for several hours. Furthermore, this increases the readiness to use the intravaginal device for the specified purpose.

Since the intravaginal device does not absorb any liquid, the vaginal region is not dried out, while the functional capability of the intravaginal device is fully retained even when it is used for several hours.

As used herein the specification and the claims, the term “intravaginal device” and related terms includes support devices, obstructing devices useful to block the flow of and/or collect bodily liquids, and the like. The term includes, without limitation, incontinence devices and vaginal supports, such as pessaries; and obstructing devices, such as menstrual collection cups and inflatable or expandable vaginal blocking devices. However, the devices, themselves, do not absorb the bodily liquids.

As used herein the specification and the claims, the term “rigidity” and related terms mean the longitudinal stability of the device. Normally specified as the unit for rigidity is the force that is necessary to compress the element in the longitudinal direction by a specific length (N/cm).

### BRIEF DESCRIPTION OF THE DRAWING

The invention is described below on the basis of schematic drawings of exemplary embodiments and graphic representations, in which:

FIG. 1 shows a longitudinal section through a first embodiment of an intravaginal device according to the invention.

FIG. 2a shows the detail II represented in FIG. 1 in the first embodiment of the intravaginal device according to the invention, represented in FIG. 1.

FIG. 2b shows the detail II represented in FIG. 1 of a second embodiment of the intravaginal device according to the invention.

FIG. 2c shows the detail II represented in FIG. 1 of a third embodiment of the intravaginal device according to the invention.

FIG. 2d shows the detail II represented in FIG. 1 of a fourth embodiment of the intravaginal device according to the invention.

FIG. 2e shows the detail II represented in FIG. 1 of a fifth embodiment of the intravaginal device according to the invention.

FIG. 2f shows the detail II represented in FIG. 1 of a sixth embodiment of the intravaginal device according to the invention.

FIG. 2g shows the detail II represented in FIG. 1 of a seventh embodiment of the intravaginal device according to the invention.

FIGS. 3a-d show side views of a number of variants of the sixth embodiment of the intravaginal device according to the invention, represented in FIG. 2f, with different patterns of holes.

FIG. 4 shows a diagram which illustrates comparative values of the rigidity of an intravaginal device according to the invention and customary pessaries.

FIG. 5 shows a diagram in which comparative values of the friction coefficient of foam and preferred sheathing materials of the intravaginal device according to the invention are entered.

### DETAILED DESCRIPTION OF THE PREFERRED EMBODIMENT

The intravaginal device may contain any resilient material that is swellable or expandable upon the introduction of water vapor or liquids, such as aqueous liquids. Such materials include absorbent materials such as fiber, foam, and the like. Preferred absorbent material for the present invention includes foam and fiber. Absorbent foams may include hydrophilic foams, foams which are readily wetted by aqueous fluids as well as foams in which the cell walls that form the foam themselves absorb fluid.

It preferably contains a pressed fibrous material which has a high degree of dimensional stability or compressive strength, in particular a high degree of axial rigidity or buckling strength, so that not only is the digital insertion of the intravaginal device into the body cavity facilitated but optimum support of the neck of the urinary bladder is also achieved over a maximum wearing period. Experimentally determined values for the rigidity are discussed below.

The fibrous material may comprise natural or synthetic fibers or of a mixture of such fibers. The natural or synthetic fibers may be smooth fibers of round cross section with a smooth surface or profiled fibers of irregular, for example stellar, cross section, which have a relatively high flexural strength.

A mixture of different fibers allows the desired physical properties of the intravaginal device, in particular its stability and its compressibility, to be set. A preferred composition comprises 75% fibers with an irregular cross section and 25% fibers with an essentially round cross section. This blend has very good expansion properties, beneficial for embodiments incorporating microporous film sheathing.

Irrespective of whether they are synthetic or natural fibers, and irrespective of whether the fibers have hydrophilic or hydrophobic properties, outstanding compaction and dimensional stability of the fibrous material can be achieved by application of heat during and/or after the compressing of the amount of fiber intended for the production of an intravaginal device. In the event that the fibers of the intravaginal device at least partially comprise bicomponent fibers, a fixed, punctiform bonding of the bicomponent fibers with other linear portions of one and the same bicomponent fiber and/or with fibers of some other composition, for example cellulose-containing fibers can be established.

By being finished appropriately for the intended purpose, the capillarity and/or absorptive capacity of the intravaginal device can be reduced or even eliminated completely. The fibrous material for the tampon core may be treated or covered with a hydrophobic material. Suitable in particular as the hydrophobic material is wax such as “Bersoft Care 6257” from the company Cognis Deutschland GmbH, Henkelstr. 67, 40551 Dusseldorf. However, the tampon core is preferably enclosed in a liquid-tight sheathing, which may comprise a film of plastic and/or a nonwoven material and/or a layer of wax.

The film of plastic and the nonwoven material may be at least partially fastened on the surface of the tampon core, for example by means of a contact adhesive or by heat sealing. Suitable in particular as the contact adhesive is a pressure-sensitive adhesive obtainable under the designation PS-34-215 from the company Stahl GmbH. The individual materials for the core and the sheathing of the intravaginal device can be selected independently of each other for achieving optimum effectiveness in each case. A layer of wax may be sprayed directly onto the core of the intravaginal device or else indirectly onto another material for the sheathing of the intravaginal device and be applied by the immersion method, depending on which purpose is to be achieved by the layer of wax. For instance, the layer of wax may form the sheathing on its own, but nevertheless at the same time form an anti-friction layer to facilitate insertion, or else be a protective layer against the penetration just of liquid or else additionally of moist gases or vapors, which is applied to a sheathing material lying under it, such as a perforated sheet of plastic or a nonwoven material for example.

According to a particularly preferred embodiment, the intravaginal device according to the invention may be used as an incontinence device, vaginal support, and/or pessary. It may then include a pressed material, preferably fibrous, which is surrounded by a nonwoven sheathing which is permeable to moist gases and/or vapors. This absorptive capacity of the intravaginal device, restricted to the moisture in gases and/or vapors, can be achieved by a selection of the fibers or other materials and the density of the nonwoven material itself and/or by a covering of the outer side of the nonwoven material with a layer of wax which is permeable to moist gases and/or vapors. This preferred intravaginal device has essentially the form of an enlarged digital menstruation tampon, so that a user can easily accept it, in particular if said user is accustomed to tampon hygiene. Again, these are especially useful for intravaginal devices used as bladder supports, pessaries, and blocking devices.

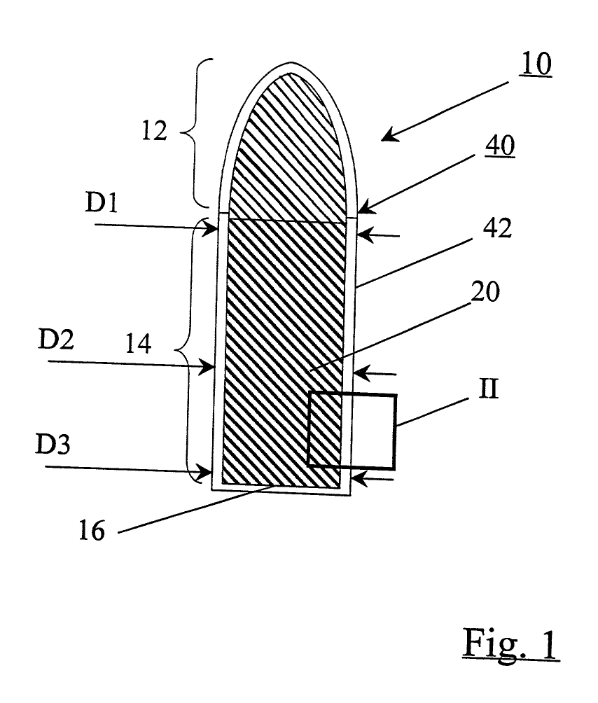

In addition, as mentioned above, intravaginal devices can also be as collection devices, such as menstrual collection cups. Therefore, as shown in FIG. 1a, the intravaginal device 30′ may include a receptacle portion 31. Other useful shapes and features may be included in such collection cup devices.

Owing to the slow moisture absorption of this preferred embodiment, the intravaginal device expands only gradually once it has been inserted into the body cavity. It is recommendable to use at least a proportion of hydrophilic or hydrophilic-finished fibers in order to accelerate the rate of absorption and expansion of the intravaginal device.

This penetration of moisture contained in gases and/or vapors through the sheathing of the intravaginal device is referred to as the moisture vapor transmission rate (MVTR), which can be controlled within wide ranges. This control of the transmission rate makes it possible to set the time which the intravaginal device requires for unrestricted, in particular radial, expansion. As a consequence, as mentioned above, the intravaginal device can be produced in a comparatively small size, in particular with a comparatively small diameter, so that the intravaginal device can be inserted into the body cavity in a manner similarly easy to that of a customary menstruation tampon.

After insertion into the vagina, the intravaginal device accepts some of the moisture present in a vaporous or gaseous state. However, the device does not accept liquids present in the vagina, and the physiological state of the vagina is essentially preserved.

For the preferred permeability of moist gases and/or vapors with a specific transmission rate, a microporous and/or microperforated structure of the sheathing material, reliably preventing the passage of liquid through the sheathing, is suitable in particular.

It is possible to use materials which are completely permeable to moisture and liquids and or microperforated and/or microporous materials which, if desired, can be set to a specific surface roughness by embossing and/or perforating for example, in order in this way to additionally assist the process of maintaining the position of the intravaginal device inserted into the body cavity.

The specific surface roughness may, however, also be achieved by means of a perforation of an outer layer of material of a multilayered sheathing, this outer layer of material preferably comprising a film of plastic. This perforated layer of material at least partially covers a further layer of material, permeable only to moist gases and/or vapors, of the sheathing, which is firmly bonded to the outer, perforated layer of material and bears with its inner side against the pressed fibrous material of the tampon core or is bonded to the latter. Edges of holes produced during the perforation are frayed to the rear side of the layer of material, so that the rear side of the perforated layer of material of the sheathing has a greater roughness in comparison with the opposite, essentially smooth, front side of the sheathing. Depending on the size of the desired friction coefficient of the surface of the intravaginal device, consequently either the smooth front side or the rough rear side with the frayed edges of the holes may be chosen as the outer side of the sheathing of the intravaginal device, because, owing to its perforation, the smooth front side of the layer of material also has a degree of roughness, which is dependent on the size of the perforation area, the distance between the holes and their diameter, as well as on the properties of the layer of material. Furthermore, a layer of material permeable to moist gases may be provided on its outer side with the layer of perforated material.

A sheathing material which is impervious to liquid water and has a moisture vapor transmission rate (MVTR) of at least about 3000 g/m²/24 h is preferred. More preferably, the MVTR is at least about 4000 g/m²/24 h. This property can be determined according to ASTM D1238.

As a result, with the customary wearing times of an intravaginal device, a controlled expansion of the intravaginal device is achieved. The radial expansion of the intravaginal device due to the take-up of moisture is in this case up to 40%, preferably approximately 20 to 30%, of the original tampon diameter.

The preferred friction coefficient of the outer surface of the tampon sheathing in comparison with a paper surface is preferably between 0.3 and 0.6, in particular between 0.35 and 0.5. Such a friction coefficient on the one hand permits unproblematic insertion of the intravaginal device, without causing irritation, on the other hand permits reliable and permanent positioning of the intravaginal device. This test can be conducted on a test apparatus from Messmer Büchel, typ k 102 (Messmer büchel, Vendelier 11, NL 3905 Veenendaal, Holland).

Before it is put to use, the intravaginal device preferably has a length of 45 to 60 mm and a diameter of 19 to 32 mm.

The rigidity or buckling strength of the intravaginal device under an axial compression of 1 cm is preferably around approximately 30 to 60 N, in particular around 35 to 45 N which can be tested in an Instron tensile testing device at a rate of 5 cm/min.

The front or insertion end of the intravaginal device is preferably sharply tapered toward its outer free end, more preferably, conically or elliptically tapered, in order to additionally facilitate the insertion of the intravaginal device into the body cavity.

According to a preferred process, the intravaginal device is produced from fibrous material by a strip of carded, randomly oriented fibrous material of a specific length, and a width corresponding at least to the length of the intravaginal device, being wound up to form a tampon blank and subsequently pressed essentially radially to form a preform. Thereafter, the pressed preform is firstly moved through a cold pressing sleeve, adapted to the profile of the pressed blank. Thereafter, the insertion end of the preform is pressed by means of a hot, dome-shaped pressing sleeve into the form of a conical tip tapered sharply toward its free end. In a subsequent process step, the preform is pressed in a hot pressing sleeve once again, in a dimensionally stable state, to form the finished intravaginal device. The residence time of the tampon in the heated tools may be adapted and set for the intended purpose according to the composition of the fibrous material used for the intravaginal device. Thus, the dimensions and temperatures of the pressing sleeves and of the forming element for the insertion end can be freely selected within wide ranges in dependence on the size and weight of the tampon core, as desired.

Under laboratory conditions, an intravaginal device according to the invention with a tampon weight of 3.6 g was radially pressed, then moved through a first cold pressing sleeve with a diameter of 16.5 mm, subsequently fed to a dome-shaped forming element with a maximum diameter of 16.5 mm, which was at a temperature of 190 C., and finally fed to a hot pressing sleeve at 170 C. with a diameter of 18.5 mm. The finished intravaginal device has a diameter of 18.3 mm in the region of its insertion end, a diameter of 19.1 mm in the region over its central length and a diameter of likewise 19.1 mm at its rear end, provided with a retrieval string.

In a second production process, in which a pressing sleeve with a diameter of 22 mm is used instead of the second pressing sleeve with a diameter of 18.5 mm, analogous diameters of the intravaginal device of 19.3 mm, 21.2 mm and 21.6 mm are obtained.

In a third production process, in which, by contrast with the second production process, the first, cold pressing sleeve has a diameter of 18.5 mm instead of 16.5 mm, diameters of the intravaginal device of 21.8 mm, 21.7 mm and 21.7 mm are obtained in the corresponding regions of its length.

The values mentioned above are average values from several measurements, which may deviate from these values in the individual case. Deviations may also occur due to the type of fibers used and due to the quality of the individual fibers. It goes without saying that dimensional differences also occur if, instead of an intravaginal device with a weight of 3.6 g, lighter or heavier devices are produced.

Depressions, preferably longitudinal grooves, are preferably made by a press in the circumferential region of an intravaginal device blank comprising a wound-up strip of nonwoven fabric. These longitudinal grooves may extend only over part of the length of the intravaginal device. In a preferred embodiment of the same, however, they extend over the entire region of the cylindrical part of the intravaginal device and at least partially into the region of the conical insertion end of the intravaginal device. A preferred incontinence device has at least eight longitudinal grooves or channels which are arranged at equal intervals in terms of the angle at circumference and extend at least over the entire cylindrical region of the intravaginal device and over approximately 50% of the axial length of the conical insertion end. While these depressions are not required, they are helpful to promote radial expansion of the product, and they can reduce the potential for wrinkling the sheathing material.

The liquid-impermeable sheathing is preferably fastened at one end of a longitudinally extending strip of a specific length of randomly oriented fibrous material before the winding up to form a wound blank and before the pressing of the same.

The sheathing may be fastened by a fastening agent, for example by means of ultrasound or a contact adhesive, but fastening by means of heat sealing is preferred. Different materials can be used as sheathing materials. Preferred are plastic materials including monolayer or multilayered plastic films. Useful materials in these films include polyolefins, such as polyethylene (PE) and polypropylene (PP), polyesters, and the like. Polyethylenes can be low density polyethylene (LDPE), linear low density polyethylene (LLDPE), and high density polyethylene (HDPE). Multilayer films can be two different films, such as an A-B construction or an A-B-A construction, or more different films, such as an A-B-C construction. The individual layers of these multilayer films can be homogenous polymeric material or blends of two or more materials. In A-B-A multilayer films, the A layers are generally the same and form substantially identical layers surrounding the B layer. The proportion of material in the A and B layers can vary as desired by the formulator. For example, the A layers can form about 90 wt-% of the film and the B layer about 10%, or the A layers can form about 65 wt-% and the B layer about 35 wt-%. The blends of polymers in an individual film or layer of a multilayer film can also vary as desired by the formulator. In many cases, a formulator of a softer film may desire to incorporate a major portion of LDPE and a minor portion of HDPE, LLDPE, PP, or one or more combinations thereof. Of course, additives can be included in the plastic film. Examples of useful films include a polypropylene (PP) film, as obtainable from the company “Clopay Plastic Products Company” under the trade name “CLOPAY P-18-3789B” and multilayer films. Prefered films combine heat sealing characteristics and a level of softness to avoid rigid portions where the film may be folded. The rigid portions are to be avoided due to potential injury during use.

It goes without saying that this film of plastic may be impermeable both to liquids and to liquid in gases and/or vapors; however, it may also be provided with more or less fine holes, to make it permeable to moisture contained in gases and/or vapors but impermeable to liquids, by means of a microperforation. This film of plastic may also be used as a roughened outer layer by means of a perforation appropriate for the intended use.

The front side of the perforated sheathing has a friction coefficient in comparison with a paper surface of approximately 0.38, while the rear side of the perforated sheathing has a friction coefficient of approximately 0.48. Both values lie in the particularly preferred range and therefore offer outstanding insertion properties, but at the same time permanently ensure correct positioning of the intravaginal device and consequently its function.

Furthermore, a paraffin dispersion obtainable under the trade name Repellan T from Cognis Deutschland GmbH, 40551 Dusseldorf, Henkelstr. 67, may be used with aluminum salts in water as a hydrophobic sheathing layer for the pressed fibrous material.

In another embodiment, a nonwoven sheathing comprising three layers is used. The first layer consists of polypropylene fibers with a basis weight of 10 g/m², the second layer consists of a mixture of 50% PET and 50% rayon, with a basis weight of 40 g/m², and the third layer, like the first layer, consists of polypropylene fibers with a basis weight of approximately 10 g/m².

Also suitable as sheathing materials which are water-impermeable but permeable to moist gases and/or vapors are microporous films, for example the microporous film “BP Chemicals PlasTec Microporous Film 31/3-11” with a basis weight of 38 g/m² and a MVTR of 3200 g/m²/24 h, or the “Witcuflex Super Dry Film Comfort Plus F-20” with a basis weight of 20 g/m².

Experiments in a moisture chamber at 40° C. and a relative atmospheric humidity of approximately 90% have shown that the rate of expansion of an intravaginal device consisting of pressed fibrous material which is completely enclosed by one of the sheathings mentioned above that are permeable to moisture in gases and/or vapors is comparable with the rate of expansion of a customary menstruation tampon sheathed with a nonwoven material. Depending on the fiber composition, the expansion is between 20 to 25% and approximately 40% over a measured time period of 1.5 h to 6 h. Nevertheless, in the case of the pessaries according to the invention, no liquid take-up and no expansion take place when they are exposed directly to a liquid.

It is further possible to construct the sheathing from two functional layers lying one on top of the other. The inner layer is the functional layer described above that is moisture-impermeable and, if appropriate, permeable to moisture in gases and/or vapors, which completely encloses the intravaginal device. Applied to this inner liquid-impermeable layer may be a second sheathing layer, with which in particular the friction coefficient is set, but which does not have to be liquid-impermeable. The outer layer may therefore completely sheath the intravaginal device, but it is also possible for this outer layer to enclose only a partial region of the intravaginal device, in particular the cylindrical outer surface, while the conical insertion end of the intravaginal device is not covered by this layer.

In this connection it may well be considered to cover a material provided with a perforation, such as a film of plastic or a layer of nonwoven material, with a layer of wax, that is to cover a material provided with a microperforation with a layer of wax of which the permeability is restricted to moist gases and/or vapors.

A comparison of the friction coefficients of preferred embodiments of an intravaginal device of the present invention with prior-art incontinence devices produced from foam is discussed below in the detailed description of the figures.

FIG. 1 schematically shows a longitudinal section through an intravaginal device 10 according to the invention, with an essentially cylindrical main body 14, a sharply conically tapered insertion end 12 and a retrieval end 16. The intravaginal device 10 comprises a tampon core 20 of compressed fibrous material, which is completely enclosed by a sheathing 40 which consists of a material which is impermeable to liquids and to moist gases and/or vapors. The material used in this embodiment for the sheathing 40 is a film of plastic 42, which completely encloses the tampon core 20 on all sides and is heat-sealed in such a way that neither liquid nor moisture can penetrate into the tampon core 20. The sheathing 40 bears directly against the tampon core 20; the distance in the schematic representation is shown merely for a visually clearer illustration of the construction. A “BP Chemicals PlasTec Microporous Film 31/3-11” from the company BP Chemicals, UK, with a basis weight of 38 g/m², may be advantageously used as the film of plastic for the sheathing.

In laboratory tests, the intravaginal device 10 has a total weight of 4.7 g and is produced in a three-stage pressing process. In a first stage, a tampon blank is pressed from a carded strip of nonwoven fabric wound up on itself in a cold pressing sleeve with a diameter of 22 mm, followed by the conical pressing of the insertion end 12 in a sleeve with a diameter of 16.5 mm at 190° C.; finally, the intravaginal device 10 is treated in a pressing sleeve with a diameter of 24 mm and at a temperature of 170° C. over a time period of 5 minutes.

The intravaginal device 10 shown in FIG. 1 has a diameter D1 of 19.8 mm in the region of the insertion end 12, a diameter D2 of 20.3 mm in the region of its main body 14 and a diameter D3 of 20.7 mm at the retrieval end 16 of the intravaginal device 10.

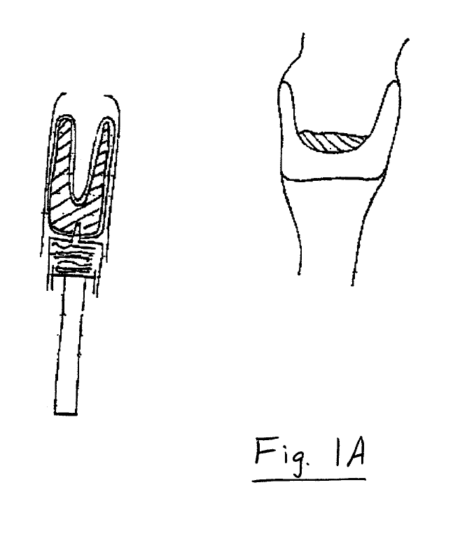

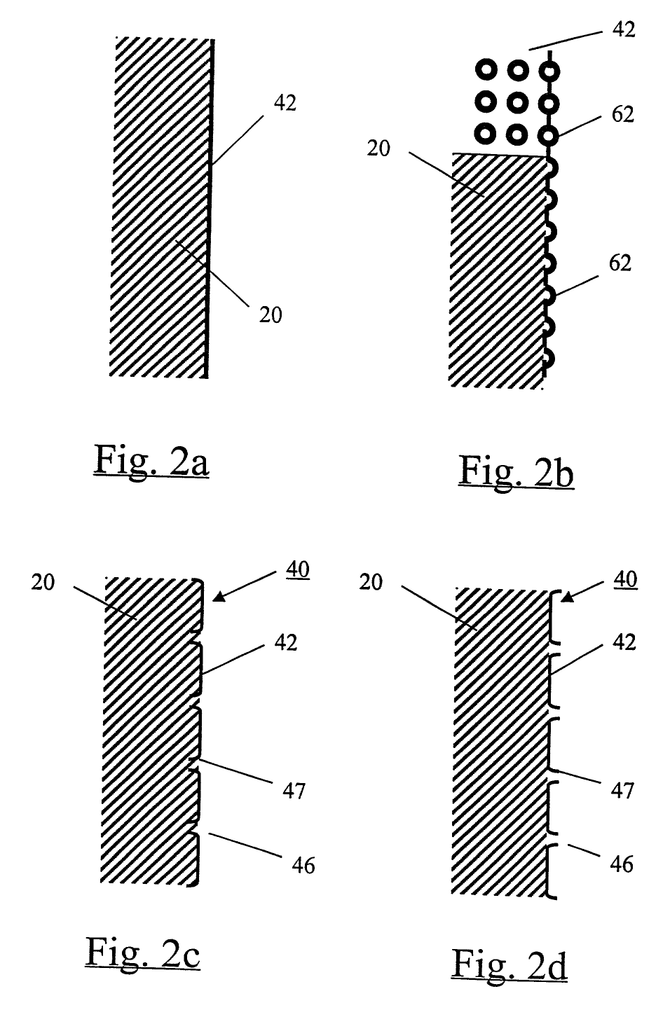

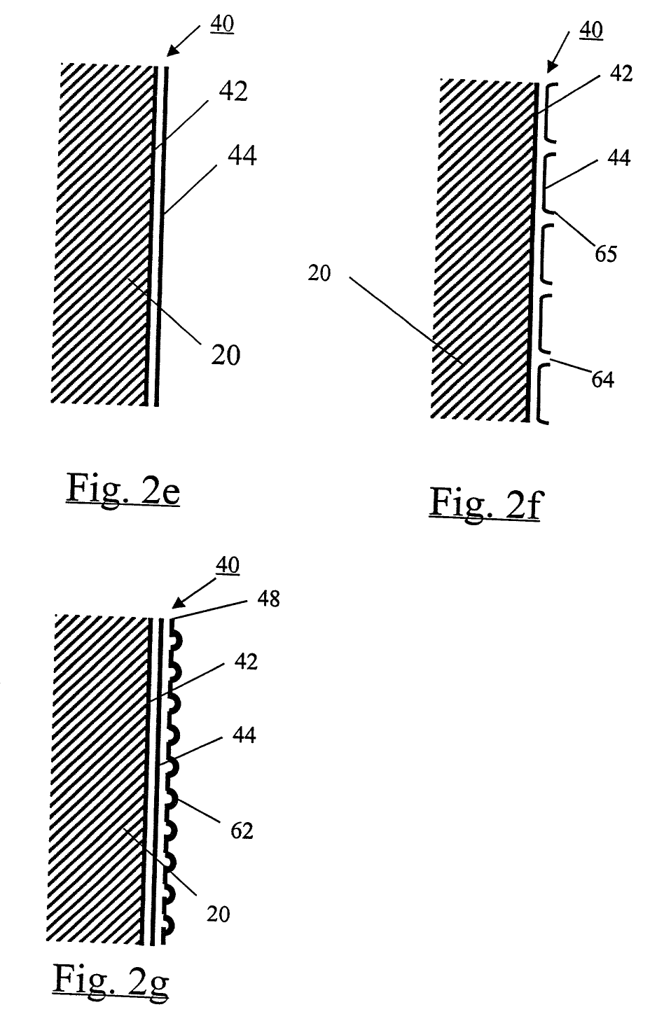

FIG. 2a shows enlarged the detail II of an embodiment of the intravaginal device 10 according to the invention shown in FIG. 1. A sheathing 42, which comprises a single layer of a film of plastic which is not permeable to liquids and completely encloses the pressed fibrous material, can be seen. The film of plastic 42 is bonded to the pressed fibrous material by heat sealing. In this embodiment, a mixture of 75% profiled fibers and 25% smooth fibers was used as the fibrous material, the profiled fibers having an essentially stellar cross section transversely with respect to their longitudinal axis.

FIG. 2b shows an analogous detail II of a second embodiment of the intravaginal device according to the invention. The same materials as in the first embodiment were used for the tampon core 20 and for the sheathing 42. However, by contrast with the first embodiment, shown in FIG. 2a, the film of plastic 42 is of a profiled form. The film of plastic 42 has embossed points 62 distributed essentially uniformly over its surface. The embossed points 62 have a diameter of less than 1 mm and an average spacing of approximately 3 mm. In this embodiment, the embossed points are formed such that they are embossed radially outward from the tampon core 20, protruding at their most elevated point only a few tenths of a millimeter above the base surface of the film of plastic 42.

For the purposes of illustration, in FIG. 2b the upper part is schematically depicted as a plan view of the detail II of the intravaginal device 10 according to the invention, while the lower part is represented as a cross-sectional view, like FIG. 2a. If appropriate, the film of plastic 42 in FIG. 2b may also be used in an inverted orientation. In this case, the embossing direction of the embossed points 62 extends radially inward toward the tampon core. The embossed points 62 made in the film of plastic 42 have the effect of increasing the friction coefficient of the intravaginal device, whereby more reliable positioning of the intravaginal device 10 is ensured even when the user is engaging in sporting activity or under increased stress.

The friction coefficient can be modified by the dimension of the embossed points 62, i.e., by the choice of the diameter and height, the spacing of the embossed points 62 from one another and finally the choice as to whether the embossed points 62 are to lie on the outside or inside.

FIG. 2c shows a detail II of a third embodiment of the intravaginal device 10 according to the invention. Here, too, the tampon core 20 consists of pressed fibrous material, which in this case comprises 90% profiled fibers and 10% smooth fibers.

The single sheathing layer, forming the sheathing 40, is again the same film of plastic 42 used in the embodiments described above, a film that is impermeable to liquids and moist gases and/or vapors. However, the film of plastic 42 has microperforations 46. The average spacing of the microperforations 46 is approximately 1.1 mm. The microperforations are so small that liquid cannot penetrate through them, but moist gases and/or vapors can, so that the tampon core 20 can take up through the microperforations 46 and absorb moisture contained in vapor or gas.

The microperforations 46 are made in the film of plastic 42 by means of very fine needles. It is also possible, however, to perform the microperforation 46 by means of other technical processes, for example by laser radiation, or by hydraulic or pneumatic means.

Edges of holes 47 produced during the microperforation 46 of the film of plastic 42 protrude beyond the plane of the film of plastic 42 inward toward the tampon core 20 and are usually frayed.

In addition to the primary effect of the microperforation 46, that of making the sheathing 40 permeable to moisture in gases and/or vapors, as a secondary effect the friction coefficient of the outer surface of the film of plastic 42 forming the sheathing 40 is increased, whereby reliable positioning of the intravaginal device 10 is assisted.

FIG. 2d shows a fourth embodiment of the intravaginal device according to the invention, which is largely identical to the third embodiment, shown in FIG. 2c, but in which the microperforations 46 of the film of plastic 42 have been applied in an inverted orientation to the tampon core 20 in comparison with the embodiment shown in FIG. 2c. As a result, the edges of the holes 47 of the microperforation 46, produced during the microperforation, protrude outward, radially with respect to the approximately cylindrical surface of the sheathing 40, causing a higher friction coefficient in comparison with the embodiment represented in FIG. 2c.

In addition to the size of the desired friction coefficient, the degree of vapor permeability or the moisture vapor transmission rate (MVTR) can be largely determined within wide limits by the density of the microperforation 46, i.e., by the number of microperforations per unit area.

The film of plastic 42 with microperforations 46 may be fastened on the tampon core 20 by means of a contact adhesive and encloses as a single sheathing layer all the regions of the tampon core 20.

Represented in FIG. 2e is a fifth embodiment of the intravaginal device 10 according to the invention. In this embodiment, the sheathing 40 around the tampon core 10 comprises two sheathing layers 42 and 44, the first or inner sheathing layer 42 comprising a material which is liquid-impermeable but permeable to moist gases and/or vapors, so that a similar effect as with the microperforated film of plastic described above is achieved. Here, too, a “BP Chemicals PlasTec Microporous Film 31/3-11” with a basis weight of 38 g/m² may be used as the microporous film of plastic 42.

Applied to the inner film of plastic 42 is a second sheathing layer 44. This layer 44 comprises a nonwoven material and forms a roughened layer with an increased friction coefficient. In addition to the reliable positioning already mentioned, this roughened layer also has the advantage that it feels more pleasant and, as a result, increases the comfort for the user.

As a departure from the embodiment described in FIG. 2e, instead of the film of plastic 42, the layer of nonwoven material 44 may also be covered on its inner side, which bears against the pressed fibrous material of the tampon core 20, with a layer of wax, which is not permeable to liquids but is to moist gases and/or vapors.

FIG. 2f shows a sixth embodiment of an intravaginal device 10 according to the invention. The inner layer 42, permeable to moisture but impermeable to liquids, is in this embodiment a layer of wax 42, but it is also possible to use the film of plastic represented in FIG. 2e. In this case as well, the roughened layer 44 is a layer of nonwoven material which has perforations 64 permeable to liquid with a diameter of in each case 2 to 3 mm, the edges of the holes 65 being directed radially outward. The layer of nonwoven material 44 is applied to the layer of wax 42 by heat sealing and is firmly bonded to the tampon core 20.

FIG. 2g shows a seventh embodiment of the intravaginal device 10 according to the invention, the sheathing 40 of which is of a three-layered construction, the inner or first layer 42 and the outer or third layer 48 respectively comprising 70% polypropylene and 30% polyethylene, while the middle layer, or the second layer, 44 comprises 70% polypropylene and 30% ethylene vinyl acetate. The thickness of the layers is chosen such that the proportion by weight of the inner layer 42 and of the outer layer 48 is in each case 25% and the proportion by weight of the middle layer 44 is approximately 50%.

In a way similar to the embodiment shown in FIG. 2b, the outer layer 48 is of a profiled form, so that it has an increased friction coefficient.

A three-layered sheathing 40 can be produced simultaneously by the extrusion process, in which the individual layers 42, 44, 48 are brought together directly after the extrusion and firmly bonded to one another before cooling down. The three-layered sheathing 40 can then be applied to the tampon core 20, for example as in the embodiment shown in FIG. 2 by means of a contact adhesive.

It is clear from the large number of embodiments represented that different sheathings 40 comprising one or more individual layers 42, 44, 48 can be made available, it being possible to set the moisture permeability or moisture vapor transmission coefficient and the friction coefficient within wide ranges according to desired requirements.

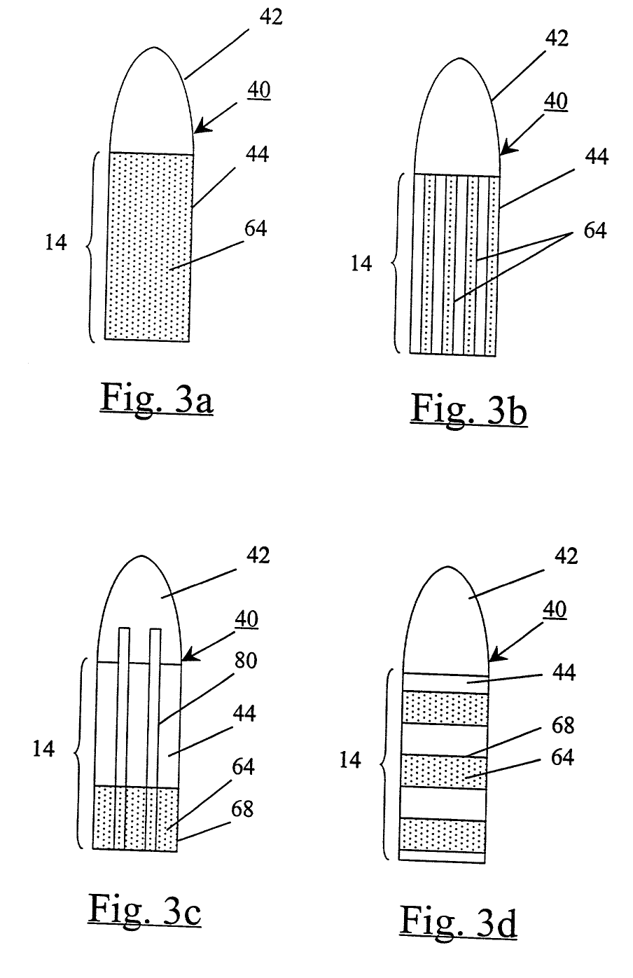

Represented in FIG. 3a is a further embodiment of the intravaginal device 10 according to the invention, in which the intravaginal device is completely enclosed by a sheathing layer 42 permeable to moist gases and/or vapors. This layer 42 serves as the inner functional layer for controlling the moisture vapor transmission, as described above.

Arranged on the cylindrical main region 14 of the intravaginal device 10 is a second, outer functional layer 44, which is at least partially perforated. This second, outer functional layer 44 is located only in the region of the main body 14 of the intravaginal device 10, has perforations 64 and merely serves for setting the friction coefficient in the desired range, in order to ensure a reliable, permanent positioning of the intravaginal device 10 in the body cavity while retaining pleasant and simple handling.

Shown in FIG. 3b is a further embodiment, which has a second outer sheathing 44, but has perforations 64, for increasing the friction coefficient and for assisting the reliable positioning of the intravaginal device 10 inserted into the body cavity, only over axially extending regions.

Schematically represented in FIG. 3c are axially extending profiled depressions in the form of longitudinal grooves 80, which extend essentially over the entire cylindrical main region 14 of the intravaginal device 10 into the conical insertion end 12 of the intravaginal device 10. These longitudinal grooves or channels 80 are produced by radial pressing by means of pressing jaws of the tampon blank described above from a wound-up strip of nonwoven fabric and enhance in particular the rigidity of the intravaginal device 10.

Although the longitudinal grooves 80 are shown only in this embodiment in FIG. 3c, all the pessaries 10 of the present invention preferably have at least eight longitudinal grooves 80 distributed uniformly over the circumference of the intravaginal device 10. Other profiled depressions may be formed instead of or in combination with the longitudinal grooves mentioned, as desired, such as, for example, punctiform, patterned or some other way.

In the embodiment shown in FIG. 3c, only an annular region 68 of the main body 14 of the intravaginal device 10 lying in the vicinity of the retrieval end 16 is provided with perforations 64, which may also be formed as microperforations.

FIG. 3d shows a further embodiment of the intravaginal device 10, the cylindrical main region 14 of the intravaginal device 10 having three annular regions 68, in which there are essentially uniformly distributed perforations 64. In addition to the desired setting or control of the friction coefficient of the intravaginal device 10, the special type of arrangement of the regions 68 having the perforations 64 allows certain visual impressions to be achieved, which may represent indications as to the type, form, size or type of use of the intravaginal device.

Instead of the perforations 64, the outer functional layer 44 may also have embossing 62, as explained in particular on the basis of the embodiment represented in FIG. 2b.

### EXAMPLES

The present invention will be further understood by reference to the following specific Examples which are illustrative of the composition, form and method of producing the intravaginal device of the present invention. It is to be understood that many variations of composition, form and method of producing the intravaginal device would be apparent to those skilled in the art. The following Examples, wherein parts and percentages are by weight unless otherwise indicated, are only illustrative.

### Example 1

A comparison was made between two prior-art incontinence devices consisting of foam, Products A and B, and an intravaginal device according to the present invention. Product A is an Incontinence tampon made of Polymer foam distributed by the company Innocept, Am Wiesenbuschl D 45966 Gladbeck, Germany under the brand name “Pro Dry”; and Product B is an Incontinence tampon made of PVA foam distributed by the company Med. SSE, Erlanger Str. 73, D 90765 Fürth, Germany.

The rigidity of the incontinence devices, which is an important factor for the digital insertion of the intravaginal device, was determined, as described above, by measuring the force necessary to compress the device, in its longitudinal direction or direction of insertion, by 1 cm. The foam devices according to the prior art are impregnated with liquid before the test, in accordance with their instructions for use. The measurements were carried out with an Instron device, model 101.

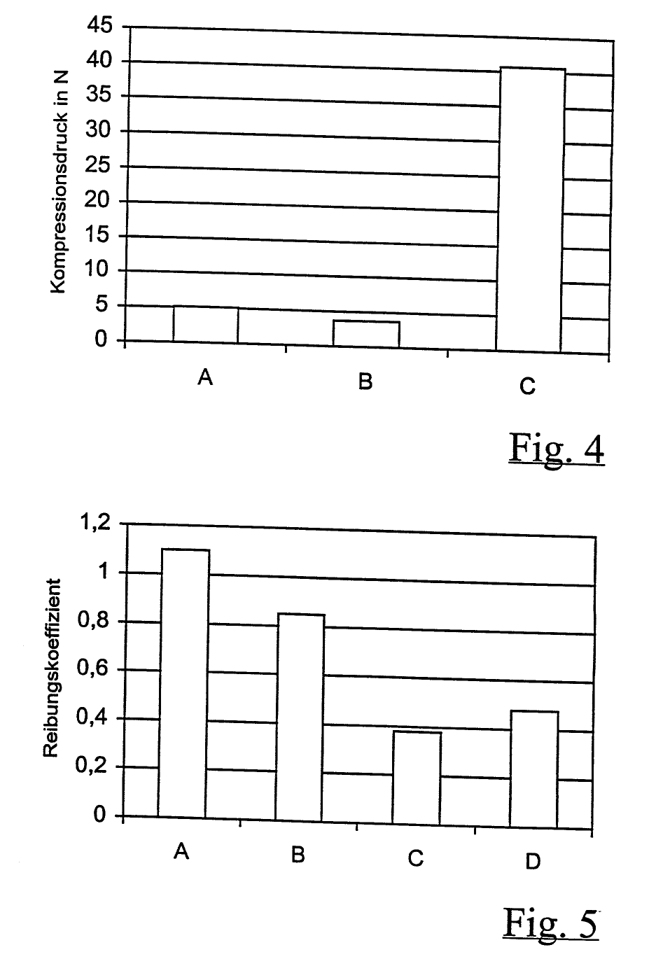

FIG. 4 shows a chart illustrating the results of this test; a comparison of the rigidity of the incontinence devices.

As the chart represented in FIG. 4 reveals, a force of only approximately 5 N has be expended for an axial compression of the incontinence devices consisting of foam by 1 cm (5.1 N in the case of product A, 3.7 N in the case of product B), while a force of over 40 N (40.7 N) is necessary in the case of the intravaginal device according to the invention of pressed fibrous material. Consequently, the rigidity of the intravaginal device according to the invention, which essentially comprises pressed fibrous material, is higher by approximately an order of magnitude than in the case of the incontinence devices of foam material available on the market, which has the consequence of significantly better insertion characteristics and better positioning properties.

### Example 2

A comparison was made between the two prior-art incontinence devices of the type tested in Example 1 and an intravaginal device according to the present invention. Products A and B are as in Example 1.

For measuring the friction coefficients of the products A and B, the foam pessaries were cut into strips. The outer sides of the flat strips served as friction surfaces. In the case of the two pessaries according to the invention, the friction coefficients were measured directly by means of samples of the sheathing surrounding the intravaginal device. The friction coefficient was determined in comparison with a paper surface; a Messmer-Büchel device was used for the measurement.

The friction coefficients of the two foam pessaries lie around 0.85 and 1.1, so that, in particular together with the very low rigidity, insertion of the intravaginal device is already made more difficult and may lead to irritation of the skin or at least to unpleasantness.

FIG. 5 shows the friction coefficients of two prior-art, commercially available incontinence devices (products A, B) in comparison with friction coefficients of two embodiments of an intravaginal device according to the invention (products E and F).

The embodiments E and F according to the invention both have a sheathing of the material described above from the Clopay Plastic Products Company with the trade name “CLOPAY P-18-3789B”, the material being provided with microperforations. In the case of the product E, the smooth front side of the sheathing forms the outer side of the intravaginal device, while in the case of the product F the rough rear side of the sheathing forms the outer side of the intravaginal device.

The friction coefficients achieved with the pessaries according to the invention, of 0.38 and 0.48, constitute an outstanding compromise between easy insertion, which requires a low friction coefficient, and reliable positioning or protection against changing of the correct position of the intravaginal device in the body cavity, which requires a higher friction coefficient.

The specification and examples above are presented to aid in the complete and non-limiting understanding of the invention disclosed herein. Since many variations and embodiments of the invention can be made without departing from its spirit and scope, the invention resides in the claims hereinafter appended.

What is claimed is:

1. An intravaginal device for feminine hygiene, wherein the intravaginal device comprises a core which is completely surrounded by a sheathing that is impervious to liquid water.

2. The intravaginal device as claimed in claim 1, wherein the core comprises compressed fibrous material.

3. The intravaginal device as claimed in claim 2, wherein the fibrous material consists of natural and/or synthetic fibers.

4. The intravaginal device as claimed in claim 3, wherein the fibrous material consists of smooth fibers and/or profiled fibers.

5. The intravaginal device as claimed in claim 1, wherein the fibrous material has at least partially hydrophilic properties.

6. The intravaginal device as claimed in claim 1, wherein the sheathing is at least partially fastened on the surface of the core consisting of pressed fibrous material.

7. The intravaginal device as claimed in claim 6, wherein the sheathing is fastened on the core by means of a contact adhesive.

8. The intravaginal device as claimed in claim 6, wherein the sheathing is heat-sealed to the core.

10. The intravaginal device as claimed in claim 1, wherein the sheathing has a MVTR of at least about 3000 g/m²/24 h.

11. The intravaginal device as claimed in claim 10, wherein the sheathing has a MVTR of greater than about 4000 g/m²/24 h.

12. The intravaginal device as claimed in claim 1, wherein one surface of the sheathing is continuously closed and smooth.

13. The intravaginal device as claimed in claim 1, wherein one surface of the sheathing is at least partially roughened.

14. The intravaginal device as claimed in claim 13, wherein the roughened surface of the sheathing is embossed.

15. The intravaginal device as claimed in claim 1, wherein the sheathing comprises at least one plastic film layer.

16. The intravaginal device as claimed in claim 1, wherein the sheathing comprises at least one nonwoven layer.

17. The intravaginal device as claimed in claim 1, wherein the sheathing comprises a layer of wax.

18. The intravaginal device as claimed in one claim 1, wherein the sheathing comprises at least two layers of material which at least partially lie one on top of the other and are bonded to one another.

19. The intravaginal device as claimed in claim 18, wherein two layers of material are present, one of the two layers of material being a roughened layer and the other of the two layers of material being impermeable to liquid water.

20. The intravaginal device as claimed in claim 19, wherein the roughened layer forms an outer surface and the liquid water-impermeable layer forms an inner surface of the sheathing.

21. The intravaginal device as claimed in claim 19, wherein the roughened layer is a layer of nonwoven material.

22. The intravaginal device as claimed in claim 19, wherein the liquid-impermeable layer is a film of plastic.

23. The intravaginal device as claimed in claim 16, wherein one surface of the layer of nonwoven material has a layer of wax disposed thereon.

24. The intravaginal device as claimed in claim 18, wherein two layers of material are present, the two layers of material being films of plastic, one of the two films of plastic of the sheathing being a perforated film provided with holes having edges which are frayed on one side of the film of plastic.

25. The intravaginal device as claimed in claim 24, wherein the side of the perforated film of plastic (44) which has the frayed edges of holes forms an outer surface of the sheathing (40).

26. The intravaginal device as claimed in claim 11, wherein the sheathing comprises an at least partially microporous layer.

27. The intravaginal device as claimed in claim 11, wherein the sheathing comprises an at least partially microperforated layer.

28. The intravaginal device as claimed in claim 1 wherein the sheathing comprises a three-layers.

29. The intravaginal device as claimed in claim 28, wherein two outer-lying layers comprise about 70% polypropylene, about 30% polyethylene and their proportion by weight is 25% of the sheathing in each case, while a middle layer comprises about 70% polypropylene and about 30% ethylene vinyl acetate with a proportion by weight of 50% of the sheathing.

30. The intravaginal device as claimed in claim 29, wherein the microperforated has a pattern of holes layer has an open area of about 25 to about 40%, a center-to-center spacing of the holes of about 0.8 to about 1.2 mm and the holes having a diameter of about 0.65 to about 0.85 mm.

31. The intravaginal device as claimed in claim 16, wherein the nonwoven material is of a multilayered form.

32. The intravaginal device as claimed in claim 28, wherein a first layer of material comprises polypropylene fiber, a second layer of material comprises a mixture of about 50% polyethylene terephthalate (PET) and about 50% rayon fibers, and a third layer of material comprises polypropylene fiber.

33. The intravaginal device as claimed claim 1, wherein the intravaginal device is capable of radial expansion to at least about 120% of an original diameter of the intravaginal device in an unexpanded state after exposure to 95% relative humidity for 240 minutes at room temperature.

34. The intravaginal device as claimed claim 33, wherein the intravaginal device is capable of radial expansion to at least about 120% of an original diameter of the intravaginal device in an unexpanded state after exposure to 95% relative humidity for 240 minutes at room temperature.

35. The intravaginal device as claimed in claim 1, wherein the intravaginal device has a length of about 55 to about 60 mm and a diameter of about 19 to about 32 mm.

36. The intravaginal device of claim 1, wherein the intravaginal device is sized to provide support to a user's urinary system.

37. The intravaginal device of claim 36 which is substantially cylindrical.

38. The intravaginal device of claim 36 which is substantially ovoid.

39. The intravaginal device of claim 36 which is substantially spherical.

40. The intravaginal device of claim 1 which further comprises a receptacle portion for collecting bodily discharges.

41. The intravaginal device as claimed in claim 1, wherein intravaginal device has an axial compressive resistance is about 30 to about 60 N under an axial compression of 1 cm.

42. The intravaginal device as claimed in claim 41, wherein intravaginal device has an axial compressive resistance is about 35 to about 45 N under an axial compression of 1 cm.

43. The intravaginal device as claimed in claim 1, wherein an insertion end of the device is tapered conically.
